(19) United States
(12) Patent Application Publication
(10) Pub. No.: US 2013/0109627 A1
(43) Pub. Date: May 2, 2013

(54) ALKYLATED SP-C PEPTOID COMPOUNDS AND RELATED SURFACTANT COMPOSITIONS

(71) Applicants: Annelise E. Barron, Palo alto, CA (US); Nathan J. Brown, Newton, MA (US)

(72) Inventors: Annelise E. Barron, Palo alto, CA (US); Nathan J. Brown, Newton, MA (US)

(73) Assignee: Northwestern University, Evanston, IL (US)

(21) Appl. No.: 13/637,904

(22) PCT Filed: Apr. 1, 2011
(86) PCT No.: PCT/US11/00591

§ 371 Date: Dec. 21, 2012

Related U.S. Application Data
(60) Provisional application No. 61/320,113, filed on Apr. 1, 2010.

(51) Int. Cl. C07K 14/00

(52) U.S. Cl. CPC C07K 14/001
USPC 514/15.5; 530/326

(57) ABSTRACT

SP-C peptoid compounds, lung surfactant compositions and related surfactant replacement therapies. Such SP-C peptoids can mimic lung surfactant protein C, and can be used in conjunction with biomimetic SP-B compounds over a range of lung surfactant compositions.

This application claims priority benefit of application Ser. No. 61/320,113 filed Apr. 1, 2010, the entirety of which is incorporated herein by reference.

This invention was made with government support under Grant No. 2 R01 HL067984 awarded by the National Institutes of Health, Grant No. BES-0101195 and Grant No. CHE-0404704 awarded by the National Science Foundation. The government has certain rights in the invention.

### BACKGROUND

Lung surfactant (LS) is a unique biomaterial that is formed in alveolar epithelium. This material is essential for proper respiration as it functions at the alveolar air-liquid interface to dramatically reduce the surface tension at end expiration, preventing the lungs from collapsing. LS is made up of ˜90% lipids and ˜10% proteins. The lipid portion of lung surfactant consists of at least 50 different lipid species with dipalmitoyl phosphatidylcholine (DPPC) being the main surface tension reducing agent along with the other saturated phospholipids. Four surfactant proteins, SP-A, SP-B, SP-C, and SP-D, are also present in lung surfactant. The two hydrophilic proteins, SP-A and SP-D, belong to the C-type collagenous lectin protein family and are primarily responsible in the host-defense activities of the airways. While the hydrophobic SP-B and SP-C proteins only constitute ˜1-2% of LS mass, they are necessary components of any exogenous surfactant replacement therapy (SRT). Inclusion with the phospholipids dramatically increases surfactant adsorption and spreading at the alveolar air-liquid interface.

The absence or inactivation of lung surfactant results in atelectasis and respiratory distress syndrome. Acute lung injury (ALI) is a more complex disease resulting from a diverse set of etiologies. Lung inflammation and alterations to endogenous surfactant result in hypoxemia and decreasing pulmonary function.

A lack or a deficiency of functional lung surfactant due to alveolar immaturity in premature infants leads to neonatal respiratory distress syndrome (nRDS). Historically, nRDS was a leading cause of infant mortality, but is now routinely treated by the airway instillation of natural surfactant extracted from animal lungs. Such an SRT is largely responsible for the dramatic decline in infant mortality rates observed in the past 20 years.

Despite the efficacy of SRT, there still exist several concerns associated with the use of natural surfactant including: cost, batch-to-batch variability, and possible infectious complications. Recent evidence has also shown that SRTs may be beneficial in the treatment of other respiratory-related disorders in adult airways; however, this would require significantly more material than current isolation procedures can produce. Concerns associated with natural SRTs as well as the limited production potential have prompted investigation towards the creation of an entirely synthetic SRT with the same efficacy as the natural material; however, this endeavor is much more complicated than first thought, largely due to the great complexity of the LS components.

Attempts have been made toward synthetic LS constituents, but mimicking SP-B and SP-C has proven difficult. SP-B is too large (79 amino acids) and structurally complex to mimic with synthetic peptides and no recombinant form of SP-B has yet been reported. SP-C, on the other hand, is relatively small (35 amino acids) and lacks any tertiary structure. However, SP-C is extraordinarily hydrophobic and has a high propensity to misfold and aggregate into inactive conformations. Recently, simplified peptide sequences and non-natural oligomers have been used to mimic the activities of both SP-B and SP-C.

One interesting approach is the use of poly-N-substituted glycines or “peptoids” for the mimicry of small proteins. Peptoids are similar to peptides except that the side chains are appended to the amide nitrogens rather than to the α-carbons. (Zuckermann, R. N., Kerr, J. M., Kent, S. B. H., and Moos, W. H. (1992) Journal of the American Chemical Society 114, 10646-10647.) This feature renders peptoids essentially invulnerable to protease degradation, making them more biostable than peptides. Despite the achirality of the N-substituted glycine backbone and its lack of hydrogen bond donors, peptoids are able to adopt extraordinarily stable, chiral helices when substituted with α-chiral, sterically bulky side chains. The ability to form stable helices makes peptoids an excellent candidate for mimicry of bioactive molecules that rely on helical structure for proper function, such as the hydrophobic LS proteins. (See, e.g., Seurynck, S. L., Patch, J. A., and Barron, A. E. (2005) Chemistry & Biology 12, 77-88; Brown, N. J., Wu, C. W., Seurynck Servoss, S. L., and Barron, A. E. (2008) Biochemistry 47, 1808 1818; and Wu, C. W., Kirshenbaum, K., Sanborn, T. J., Patch, J. A., Huang, K., Dill, K. A., Zuckermann, R. N., and Barron, A. E. (2003) Journal of the American Chemical Society 125, 13525 13530.)

As discussed above, SP-C is a 35 amino acid lipopeptide that is highly conserved amongst all mammalian species and is unique to lung surfactant. The high rate of sequence conservation and specificity to lung surfactant suggest not only a critical role in surfactant homeostasis, but also that its unique sequence is paramount to its biophysical activity. SP-C's secondary structure is dominated by a 37 Å-long α-helix that stretches from residue 9 to 34. This valyl-rich helical region governs SP-C's disposition in and association with the phospholipids of lung surfactant. Despite the high rate of sequence conservation of this region, it is thought that the helical conformation itself, rather than the precise covalent structure, is of prime importance for the biological function of synthetic SP-C analogues. This strategy allows for the replacement of the problematic, aggregation prone sequence with side chain structures that are more stable, allowing for the efficient production of a synthetic SP-C analogue.

A similar conclusion was found in peptoid analogues of SP-C in which a peptoid mimic with an α-chiral, aromatic helix displayed superior surface-active features in comparison to a more biomimetic peptoid mimic with an α-chiral, aliphatic helix. (See, Brown, N. J., Wu, C. W., Seurynck-Servoss, S. L., and Barron, A. E. (2008) Biochemistry 47, 1808-1818.) The difference in surface activity. between the two mimics was attributed to the increased structural rigidity imparted in the helical region by the bulkier aromatic groups.

As illustrated in the foregoing, it has proven problematic to optimize side chain chemistry, helical rigidity and/or overall surface activity. As a result, there remains an on-going concern in the art to provide an alternative approach to biomimetic SP-C compounds of the sort for use in pulmonary surfactant compositions, associated surfactant replacement therapies and/or, generally, for treatment of respiratory distress.

### SUMMARY OF THE INVENTION

In light of the foregoing, it is an object of the present invention to provide biomimetic peptoid-related SP-C compounds, related compositions and/or methods of treatment using such compounds and/or compositions, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It can be an object of the present invention to provide a range of biomimetic peptoid and peptoid-peptide SP-C compounds varied by residue, side-chain identity and/or sequence, such compounds as can be considered by structure and/or confirmation, resulting surface activity and affect on overall function, as can be distinguishable over SP-C components of the prior art.

It can also be an object of the present invention to incorporate one or more SP-C compounds of this invention into a range of lung surfactant compositions, alone or together with an available SP-B component, to affect and/or enhance surface activity at an air-liquid interface.

It can also be an object of the present invention, alone or with consideration of one or more of the foregoing objectives, to provide such a biomimetic, peptoid-related SP-C compound and/or related lung surfactant composition for in vivo treatment of a mammalian pulmonary disorder.

Other objects, features, benefits and advantages of the present invention will be apparent from this summary and the following descriptions of certain embodiments, and will be readily apparent to those skilled in the art having knowledge of various lung surfactant protein components, biomimetic peptoid compounds and related synthetic techniques. Such objects, features, benefits and advantages will be apparent from the above as taken into conjunction with the accompanying examples, data, figures and all reasonable inferences to be drawn therefrom, alone or with consideration of the references incorporated herein.

In part, the present invention can be directed to a poly-N-substituted glycine lung surfactant compound comprising a helical, hydrophobic C-terminal component comprising at least 12 N-substituted glycine residues; and a non-helical N-terminal component comprising at least one N-substituted or alkyl-substituted glycine residue, where such an alkyl substituent can be selected from about C₄ to about C₂₄ linear, branched and cyclic moieties. As described or referenced elsewhere herein, such N-substituents can be independently selected from α-amino acid side chain moieties and structural/functional analogs thereof and proline residues. As described elsewhere herein, such a sequence can provide such a compound a certain amphipathicity. Such structural and/or functional analogy can be considered in the context of any such α-amino acid side chain, N-substituent and/or sequence of such N-substituted glycine residues, such structure and/or function including but not limited to charge, chirality, hydrophobicity, amphipathicity, helical structure and facial organization. Such analogs include, without limitation, carbon homologs of such side chains—such homologs including but not limited to plus or minus one or two methylene and/or methyl groups.

In certain such embodiments, the N-terminal component of such a compound can comprise one or two such N-substituted glycine residues. In certain such embodiments, such a compound can comprise two such residues. Without limitation, an N-terminal component can comprise two adjacent N_{oct}(oct≡octadecyl) residues.

As illustrated herein, such N-substituted glycine residues can be considered in conjunction with an N_{sub} designation, where “sub” refers to an N-pendant substituent, such a substituent as can be an α-amino acid side chain, alkyl, or any other substituent described or referenced elsewhere herein. Such N-substituted glycine residues, substituents and corresponding designations are as would be understood by those skilled in the art made aware of this invention, for instance as shown in U.S. Pat. No. 6,887,845, the entirety of which is incorporated herein by reference.

In part, this invention can be directed to a poly-N-substituted glycine compound of a formula

A-X—Y—Z—B

wherein A can be an N-terminus and can be selected from H and 1-2 N-alkyl substituted glycine residues, where such an alkyl substituent can be selected from or can comprise about C₄— about C₂₄ linear, branched and cyclic alkyl moieties; X can be a component comprising residues selected from 1-about 7 N-substituted glycine residues and proline, and combinations thereof; Y can be a component comprising 1-about 6 N_{Lys} residues; Z can be a component comprising about 12-about 20 N-substituted glycine residues, such a Z component as can comprise residues selected from Nₛₚₑ, N_{sdp} and N_{ssb} residues, and combinations thereof; and B can be a C-terminus as can be selected from NH₂ or another available C-terminal moiety reflective of residue sequence or synthetic technique (e.g., —COOH, etc.), and 1-2 N-substituted glycine residues selected from N_{sdp} and Nₛₚₑ.

In certain non-limiting embodiments of such a compound, A can be selected from N-alkyl substituted glycine residues. In certain such embodiments, each such alkyl substituent can be independently selected from linear C₈-C₂₀ alkyl moieties. Regardless, in various such or other embodiments, X can comprise an Nₛₚₑ residue. In certain embodiments, such an X component can be a sequence of about 3-about 5 residues. In certain such non-limiting embodiments, at least one such residue can be Nₚₘ and/or at least one such residue can be proline. Regardless of X or Y, such a Z component can be a sequence of about 10-about 16 residues comprising a combination of N_{sdp} and Nₛₚₑ residues. Without limitation, in certain such embodiments, at least about ⅔ of such a Z component can be N_{sdp} residues. In certain other embodiments, Z can be a sequence of about 12-about 14 N_{sdp} residues.

In yet other non-limiting embodiments, such X and Z components can independently comprise at least one other residue selected from proline and N-substituted glycine residues, such N-substituents independently as can be selected from α-amino acid side chain moieties and carbon homologs thereof. In certain such embodiments, X and Z can independently comprise residues selected from Nₚₘ, N_{ssb}, N_{sdp}, N_{Leu}, N_{Ile}, N_{Phe}, N_{Trp}, N_{His} and N_{Tyr}, and combinations thereof. In certain such embodiments, Y can comprise an N_{Arg} residue. Regardless, such an X component can be a sequence of about 3-about 5 residues, a Y component can be a sequence of about 2-about 3 residues, a Z component can be a sequence of about 10-about 14 residues, and B can be NH₂ or another available C-terminal moiety reflective of residue sequence or synthetic technique (e.g., —COOH, etc.).

In part, the present invention can be directed to a poly-N-substituted glycine lung surfactant compound of a formula

A-X—Y—Z—B

wherein A can be an N-terminus and can be selected from H and 1-2 N-substituted glycine residues, where such an alkyl substituent can be selected from about C₄ to about C₂₄ linear moieties; X can be a component comprising residues selected from about 3-about 5 N-substituted glycine residues and proline, and combinations thereof; Y can be a component comprising residues comprising about 2-about 3 N_{Lys} residues; Z can be a helical component comprising about 10-about 14 N-substituted glycine residues, such residues comprising residues selected from Nₛₚₑ and N_{sdp} residues, and combinations thereof; and B can be a C-terminal moiety of such a compound, including but not limited to NH₂.

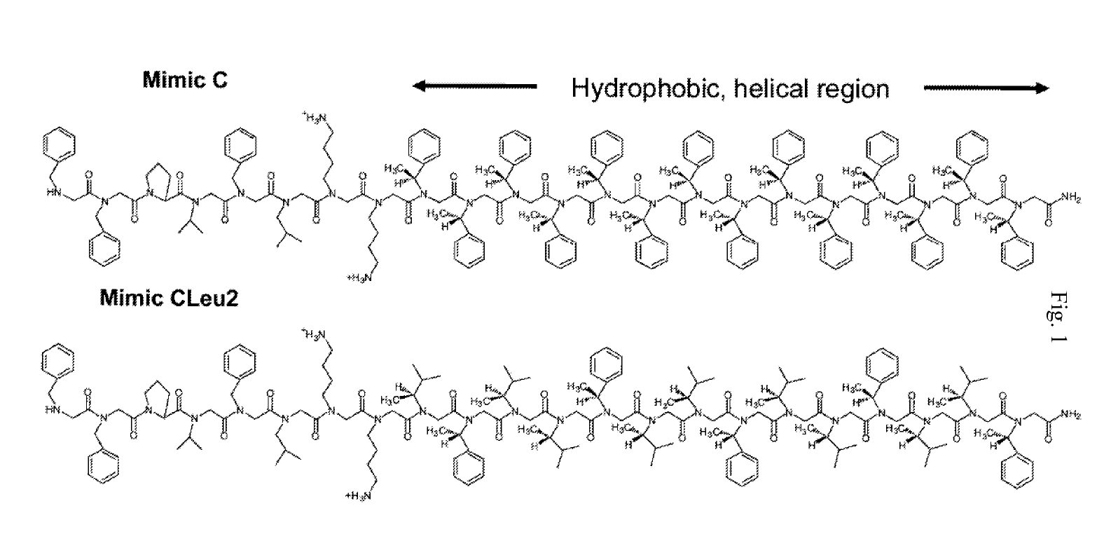

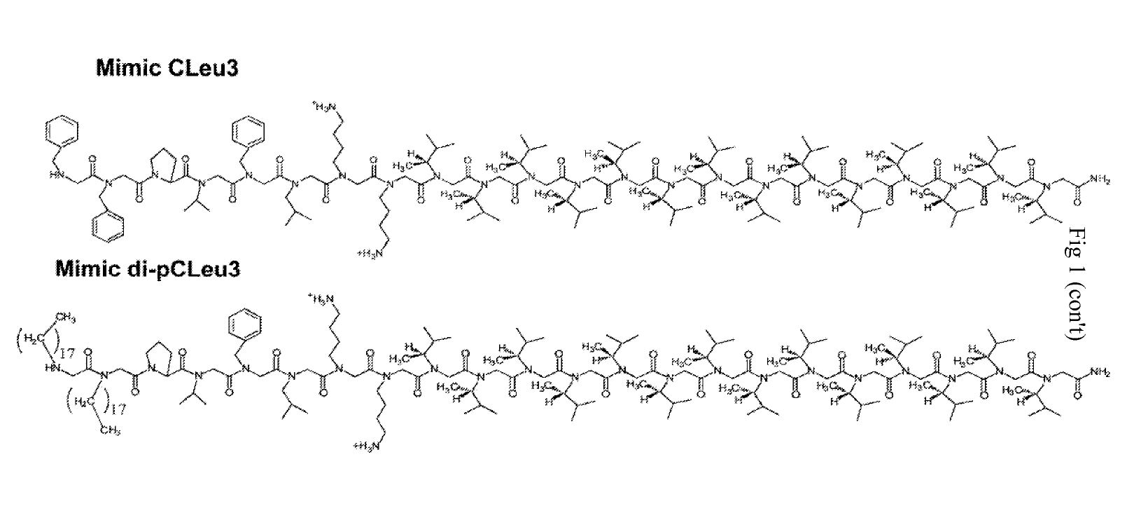

More generally, with consideration of the preceding, the peptoid-related compounds of this invention can comprise about 15 to about 35 residues. Such compounds can be alkylated (e.g., including one, two or more N-alkyl substituted glycine residues) or non-alkylated at, about or near the N-terminus. Notwithstanding sequence length or alkylation, the helical C-terminal section of such a compound can comprise up to about 20 Nₛₚₑ residues (see, e.g., FIG. 1), one or more of which can be independently interchanged with an Nₚₘ (pm≡phenylmethyl) residue, an N_{ssb} (ssb (S)-sec-butyl) residue, an N_{sdp} (sdp≡(S)-1,2-dimethylpropyl) residue or a residue comprising an N-leucine (Leu), isoleucine (Ile) or phenylalanine (Phe) substituent or a substituent structurally and/or functionally equivalent thereto.

In certain non-limiting embodiments, the C-terminal component can comprise residues selected from Nₛₚₑ and N_{sdp} residues, providing at least one such residue is Nₛₚₑ. In certain such embodiments, such a component can be comprised entirely of N_{sdp} residues, regardless of overall sequence length. Without limitation, such embodiments can comprise an N-terminal component comprising at least one N-alkyl substituted glycine residue, where each such alkyl substituent can be independently selected from about C₄ to about C₂₄ linear moieties. In certain such embodiments, one or more such residues can be an N_{oct} residue. Without limitation, representative SP-C peptoid compounds of this invention are shown in FIG. 1.

In part, the present invention can also be directed to a pulmonary surfactant composition. Such a composition can comprise one or more of the poly-N-substituted glycine compounds of this invention, such a compound of the sort described herein or as would otherwise be understood by those skilled in the art made aware of this invention; and a lipid component as can comprise components selected from naturally-occurring phospholipids, non-natural analogs of such phospholipids, naturally-occurring fatty acids, non-natural analogs of such fatty acids, commercially available surface-active agents and combinations thereof. Such a composition can have or provide in vitro surface activity, physiological alveolar surface activity and/or in vivo efficacy in the treatment of a mammalian subject.

In certain non-limiting embodiments, such compositions can comprise one or more other surfactant protein components including but not limited to one or more naturally-occurring surfactant proteins or biomimetic peptoid compounds (e.g., without limitation, SP-B, one or more biomimetic SP-B components and/or one or more functional analogs thereof) such compounds now or hereafter known in the art, including but not limited to peptoid compounds of the sort described in co-pending application serial no. PCT/US2011/000511 filed Mar. 21, 2011, the entirety of which is incorporated herein by reference.

Accordingly, this invention can be directed to a range of pharmaceutical compositions comprising one or more of the present peptoid-related compounds, one or more lipid and/or phospholipid components of the sort known in the art and/or a pharmaceutically-acceptable carrier. Such compositions can be prepared and/or formulated as would be understood by those skilled in the art made aware of this invention. One or more of the peptoid compounds of this invention, alone or together with one or more naturally-occurring and/or derived surfactant proteins, protein mimics, spreading agents or structural/functional analogs thereof, can comprise about 1 wt % or less to about 20 wt % or more of such a composition, such an amount at least partially sufficient to affect and/or reduce an alveolar or in vitro air/liquid surface tension. Regardless, as illustrated below, any of the present peptoid compounds and/or related compositions can be used alone or in combination, in conjunction with one or more respiratory therapies or treatment methodologies. Without limitation, such a method can comprise providing one or more such peptoid compounds and/or related compositions; and administering such compound(s)/composition(s) using any recognized delivery technique and/or contacting a lung/alveolar interface or an in vitro air/liquid model thereof.

Accordingly, the present invention can also be directed to a method of treating a mammalian pulmonary disorder. Such method can comprise providing a mammalian subject exhibiting a physiological condition comprising a lung surfactant deficiency; and administering a composition of this invention to such a subject. Such a composition can be formulated to provide a therapeutically effective amount thereof, as would be understood by those skilled in the art made aware of this invention. In certain embodiments, together with a biomimetic SP-C compound, such a composition can comprise a component selected from naturally-derived SP-B components, biomimetic SP-B components and combinations thereof. In certain such non-limiting embodiments, such an SP-B compound can be selected from one of those described herein. Regardless, such a composition can be formulated as a liquid bolus, an aerosol spray or otherwise as understood in the art. Regardless, such a composition can be administered tracheally to such a mammalian subject.

### DETAILED DESCRIPTION OF THE DRAWINGS

FIG. 1. In accordance with this invention, chemical structures of non-limiting SP-C peptoid mimic compounds. (Mimic C is a comparative peptoid of the prior art.)

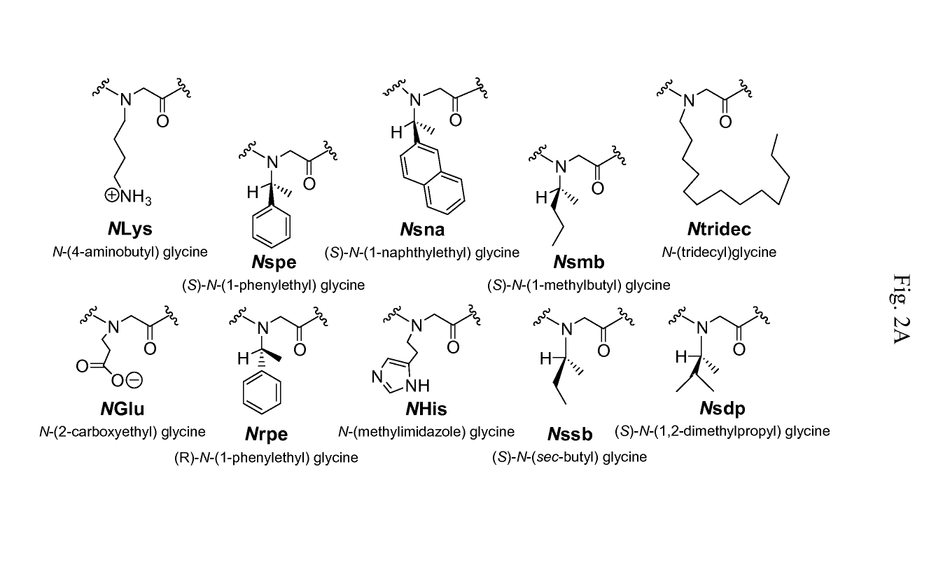

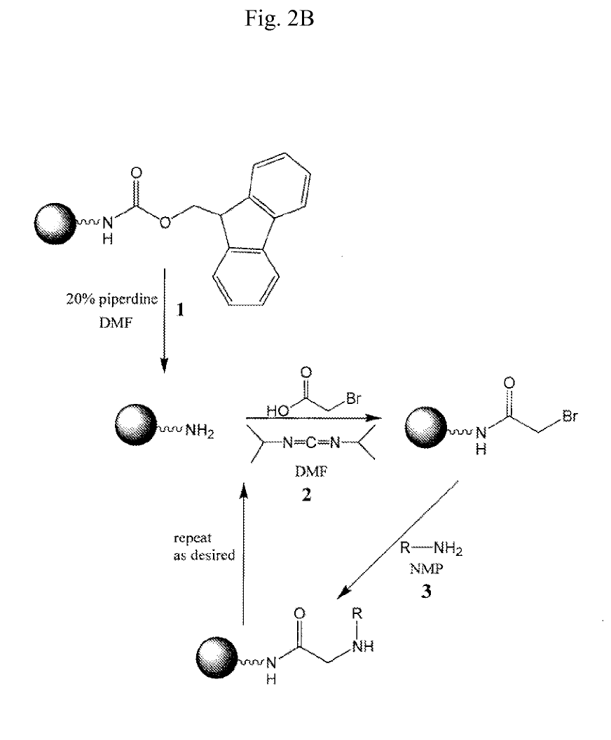

FIGS. 2A-B. In accordance with this invention, (A) various non-limiting side chain moieties, as can be incorporated into N-substituted glycine residues and corresponding peptoid compounds and related compositions; and (B) a schematic illustration of the sub-monomer synthetic protocol for polypeptoids. Steps 2 and 3 are simply repeated for the addition of each monomer unit. Once the full polypeptoid has been synthesized, it is cleaved off the resin with trifluoroacetic acid and purified by reversed-phase HPLC.

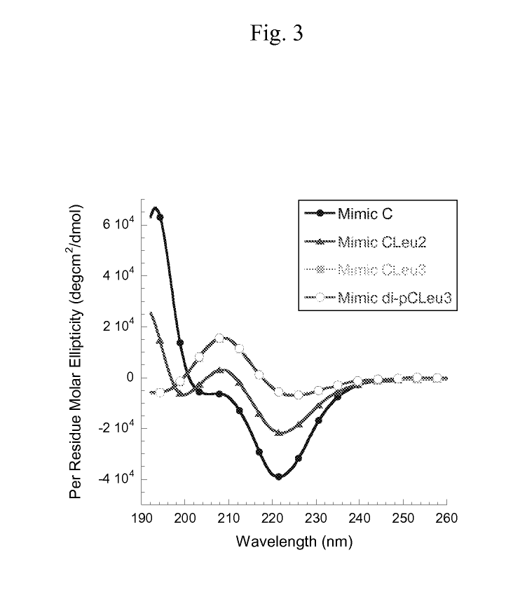

FIG. 3. Circular dichroism (CD) spectra of the peptoid-based SP-C mimics showing qualitatively similar characteristics of peptoid helices. As the aliphatic content is increased, the CD spectra display features that are progressively similar to a polyproline type I peptide helix. Spectra were acquired in methanol at a concentration of ˜60 μM at room temperature.

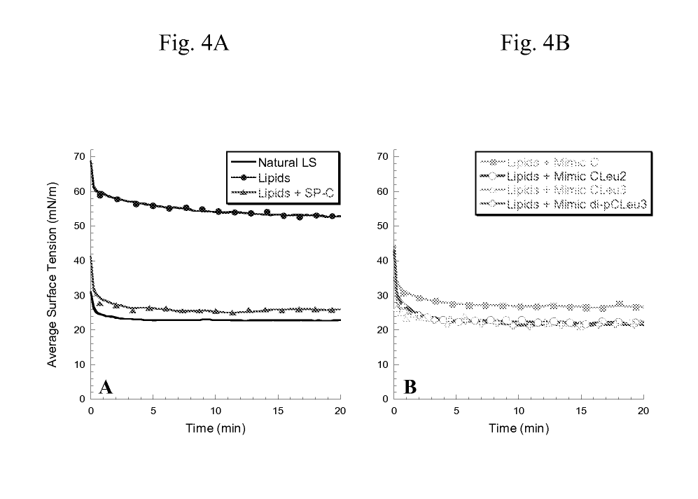

FIGS. 4A-B. Static pulsating bubble surfactometry (PBS) results displaying surface tension as a function of time. (A) Natural lung surfactant and lipid mixture alone and with 1.6 mol % SP-C; (B) Lipid mixture with 1.6 mol % Mimics C, CLeu2, CLeu3, and di-pCLeu3. Measurements were taken at a bulk surfactant concentration of 1 mg/mL lipids and at 37° C.

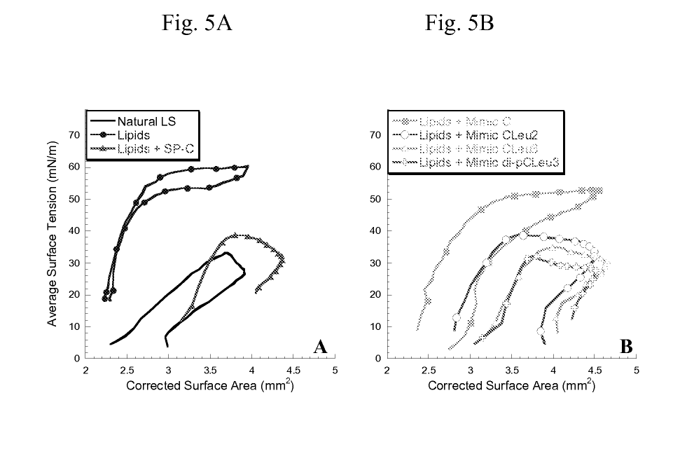

FIGS. 5A-B. Dynamic pulsating bubble surfactometry (PBS) results displaying surface tension as a function of surface area at an oscillation frequency of 20 cycles/min. (A) Natural lung surfactant and lipid mixture alone and with 1.6 mol % SP-C. (B) Lipid mixtures with 1.6 mol % Mimics C, CLeu2, CLeu3, and di-pCLeu3. Measurements were taken at a bulk surfactant concentration of 1 mg/mL lipids and at 37° C.

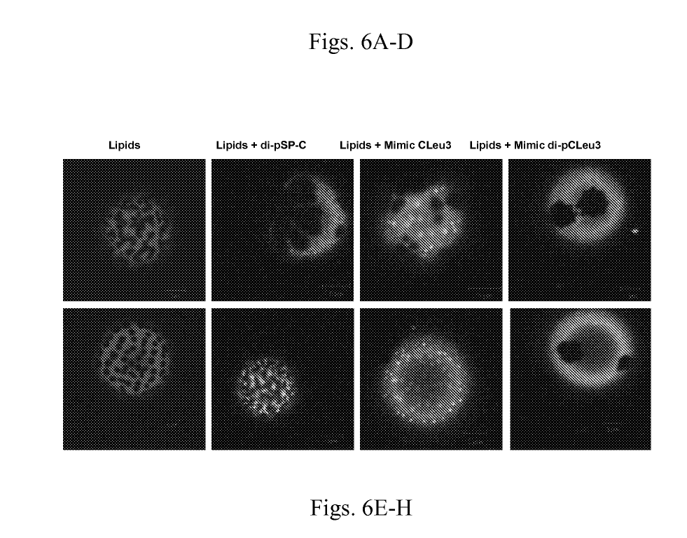

FIGS. 6A-H. Top row, panels A-D, bottom row, panels E-H. Confocal fluorescence microscopy imaging of giant unilamellar vesicles (GUVs) of lipids alone (A and E), lipids with 10 wt % di pSP C (B and F), lipids with 10 wt % Mimic CLeu3 (C and G), and lipids with 10 wt % Mimic di-pCLeu3. Scale bars correspond to 5 μm. Fluorescence images were taken at 21° C. and are representative of the population observed. Red corresponds to the fluorescently labeled lipid species and yellow to the labeled peptide and peptoids.

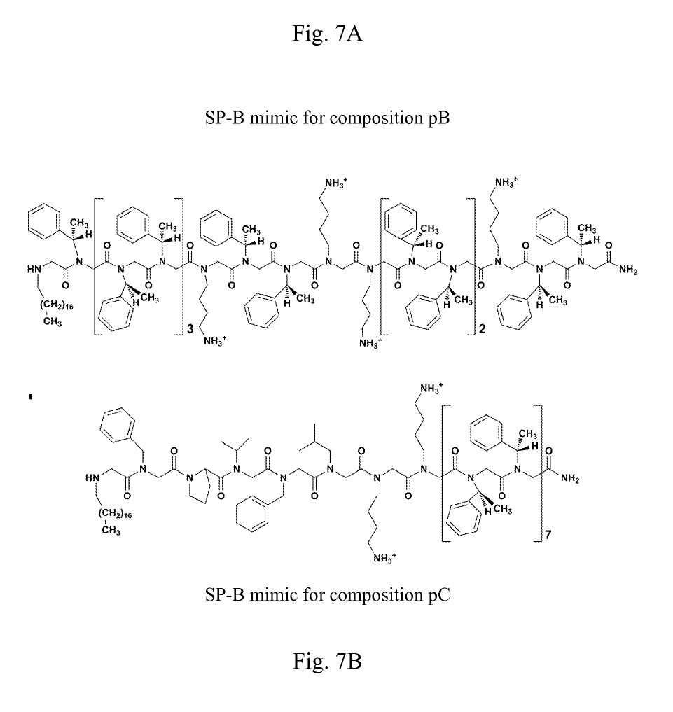

FIG. 7. Chemical structures of representative, non-limiting peptoid based mimics of SP-B and SP-C. The eight N-terminal residues of the SP-C component contain side chains that are analogous to SP-05-12, and the remaining 14 aromatic hydrophobic residues form a helix that mimics the membrane spanning, hydrophobic helix of native SP-C. The N-terminal octadecyl amine moiety of the SP-C peptoid is a motif intended to mimic the post translational modification of palmitoylated residues 5 and 6 in human SP-C. The SP-B mimic was designed to emulate the insertion region and helical amphipathic patterning of SP-B 1-25, with the added feature of an N-terminal octadecylamine substituent.

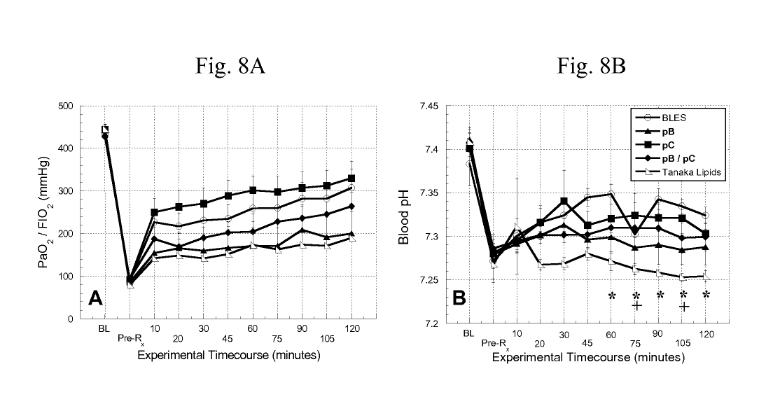

FIG. 8A-B. Physiological indicators of pulmonary gas exchange function over time. (A) PaO₂/FIO₂ and (B) Blood pH over the time course of the experiment. Error bars indicate the standard error of the mean (SEM). Statistical significance indicators: * indicates p<0.05 between BLES treatment group and Tanaka Lipids (TL); +indicates p<0.05 between pC treatment group and Tanaka Lipids.

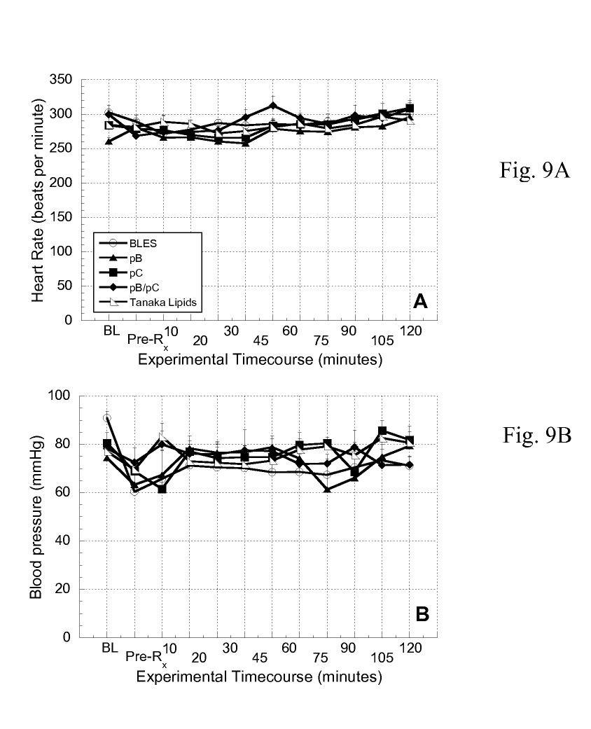

FIGS. 9A-B. Vital signs of all animals throughout the timecourse of the experiment. (A) Heart rate and (B) blood pressure at baseline measurement (BL), after lavage and before exogenous surfactant treatment (Pre-Rx), and at time points throughout the ventilation period. Error bars indicate the standard error of the mean (SEM).

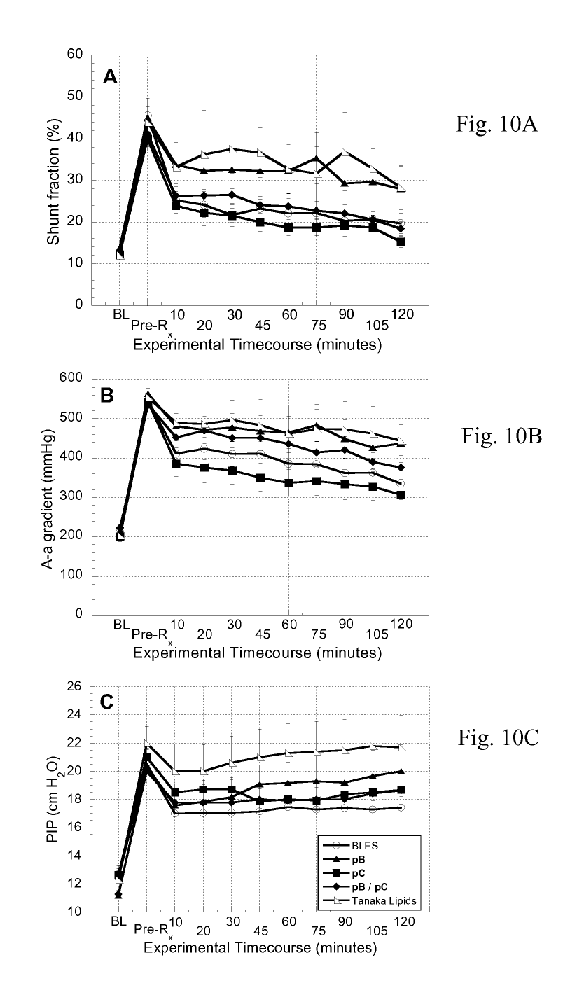

FIG. 10A-C. Physiological indicators of pulmonary function. (A) Shunt fraction (B)-A-a gradient, and (C) Peak inspiratory pressure (PIP) over the time course of the experiments. Error bars indicate the standard error of the mean (SEM). Statistical significance indicators: +indicates pC different from Tanaka lipids; * indicates BLES different from Tanaka lipids; # indicates pC different from pB.

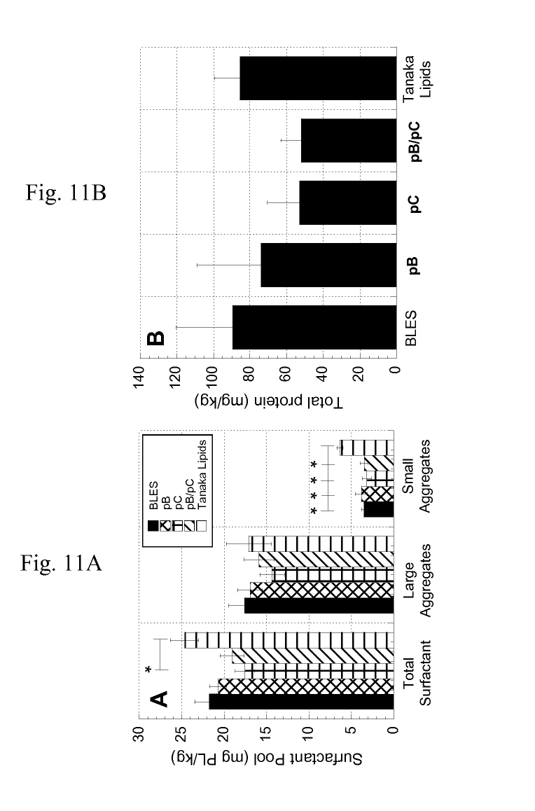

FIGS. 11A-B. Surfactant pool characterization in broncheoalveolar lavage (BAL). (A) Average amounts of total surfactant, large aggregates, and small aggregates in BAL. (B) Average total protein content in the BAL of each treatment group. Error bars indicate the standard error of the mean. Statistical significance indicators: * indicates p<0.05 for the difference between the designated group and TL alone group.

### DETAILED DESCRIPTION OF CERTAIN EMBODIMENTS

As demonstrated below, the poly-N-substituted glycine compounds of this invention, surfactant compositions and/or related methods of treatment can improve upon existing therapies and associated deficiencies. Without limitation, as compared to the prior art, various such compounds of this invention can have greater surface-adsorptive properties in a lipid film, can have greater surface-tension-reducing properties in a lipid film, and/or can be varied by residue sequence and/or N-substituent—to provide improved hydrophobicity and/or amphipathicity and/or lipid affinity.

In support thereof, representative peptoid compounds were prepared as described in Example 1 and illustrated in FIG. 2B. Variations on such synthetic techniques, N-substituents, resulting glycine residues and corresponding peptoids and related peptoid-peptides are as described in co-pending applications PCT/US2011/000511 (filed Mar. 21, 2011), Ser. No. 12/378,034 (filed Feb. 9, 2009) and U.S. Pat. No. 6,887,845—each of which is incorporated herein by reference in its entirety

Various non-limiting embodiments of this invention represent continued work on structure-activity relationships in peptoid-based analogues of SP-C. Peptoid analogues that emulate the extreme hydrophobicity, highly helical structure, and longitudinal amphipathicity of SP-C are able to provide many of the biophysical properties of SP-C. Side chain chemistry and structural rigidity of the C-terminal helical region, along with a hydrophobic N-terminal region, have been shown important for the biophysical activity of such peptoid-based mimics. Building upon such structural insights, the present invention adopts a new helix-inducing side chain: N—S-1,2-dimethylpropylamine (Nsdp) was utilized to better combine the structural and biomimetic sequence requirements of the hydrophobic helix in a single side chain structure. Peptoid mimics incorporating varying amounts of Nsdp in the helical region were synthesized and subsequently characterized. An alkylated version of a mimic containing solely Nsdp residues in the helical region was also studied as this modification greatly increases the surface activity of peptoid-based SP-C mimics. The secondary structures of the peptoid SP-C mimics were assessed in organic solution using circular dichroism (CD) spectroscopy. Pulsating bubble surfactometry (PBS) and fluorescence imaging of giant unilamellar vesicles (GUVs) were used to characterize the static and dynamic surface activity and film morphology of the mimics in a biomimetic phospholipid formulation. The results show that the rationally designed peptoid analogues are able to fully replicate not only the biophysical activity of native SP-C, but also display similar compression-expansion behavior as natural lung surfactant containing both SP-B and SP-C proteins. These studies provide strong evidence that peptoid analogues of SP-C incorporating various and varying number of N-alkylamine and/or Nsdp residues hold significant promise for use in a synthetic surfactant therapy for the treatment of respiratory illnesses.

Illustrating certain such embodiments, varying numbers of the α-chiral, aliphatic side chain Nsdp residues were incorporated in the helical region (FIG. 1). This side chain contains an extra methyl branch as compared to the previously studied α-chiral, aliphatic side chain, N—(S)-sec-butylamine (Nssb), and was considered as a route to strengthen the helical structure—presumably, due to the added steric repulsion. Accordingly, four peptoid-based analogues were studied: Mimic C, Mimic CLeu2, Mimic CLeu3, and Mimic di-pCLeu3. Mimic C contains an entirely α-chiral, aromatic peptoid helix that was previously shown to mimic the surface properties of a SP-C peptide well and is used here as a comparator species. Mimic CLeu2 is similar to Mimic C except that the helical region contains approximately two-thirds of the α-chiral, leucine-like, Nsdp side chain and one-third α-chiral, aromatic residues. Because of the periodicity of the peptoid helix and the placement of the side chains, Mimic CLeu2 is predicted to have two aliphatic faces and one aromatic face in the helical region. Mimic CLeu3 contains a helical region that is entirely composed of the α-chiral, aliphatic Nsdp side chains and is closest to the native SP-C helix composition. An alkylated version of Mimic CLeu3 was also studied (Mimic di-pCLeu3), to determine whether N-terminal alkylation would improve the dynamic surface activity of such SP-C analogues.

In addition to being more sterically bulky, the Nsdp side chain is also more similar to valine and leucine side chains normally present in the SP-C helix. The structural differences between a peptoid polyproline type I-like helix and a standard peptide α-helix cause the side chains to project out at different angles. This difference in orientation and projection in the elongated peptoid structure ˜6-6.7 Å per turn) can be overcome by the placement of an extra methylene group in the side chain, making Nsdp very similar to a valine side chain.

Circular Dichroism.

CD was used to characterize and compare the secondary structures of the peptoid analogues in solution. SP-C and synthetic SP-C peptide mimics form predominantly α-helical structures in solution, which correlate strongly with their surface activity. CD spectra of the modified of the peptoid-based SP-C mimics, are displayed in FIG. 3.

CD features that are similar to a peptide α-helix with an intense maximum at λ˜192 nm and double minima at λ˜205 nm and ˜220 nm (FIG. 3). These spectral features are characteristic signatures of a helical peptoid structure in oligomers of this class with highly ordered backbone amide bonds. Adding two Nsdp faces in the helical region results in a slightly different type of CD spectrum for Mimic CLeu2. Mimic CLeu2 displays spectral features that are more similar to a polyproline type I peptide helix with a shallower minimum at λ˜220 nm and a shifted local minimum at λ˜200 nm. The spectrum for Mimic CLeu2 is very similar to that observed for a mixed aromatic and aliphatic mimic previously studied, although, the intensity is increased, indicating a more confined, helical structure. Mimic CLeu3 with only α-chiral, aliphatic residues in the helical region, displays a slightly different CD spectrum. These CD spectral features that are similar to those of a polyproline type I peptide helix, but blue-shifted, which is characteristic of peptoids of this class. Similar to Mimic CLeu2, t CD spectrum for the entirely Nsdp mimic is also more intense than that of the corresponding Nssb-based mimic. The addition of the amide-linked alkyl chains in the N-terminal region does not alter the CD spectrum of Mimic di-pCLeu3. This is consistent with other peptoid analogues of SP-C where only a minimal change in CD spectrum was observed. Despite differences in the type of CD spectra observed for the various compounds, all of the SP-C mimics are shown to be helical and structured in solution, satisfying one of the structural criteria, helicity, believed to be of importance for the surface activity of SP-C. In addition, the spectra for the peptoid-based mimics did not appreciably change over time, indicating the analogues are stable and not prone to aggregation in solution like the native protein.

Static Bubble Adsorption.

One of the key biophysical characteristics that is important to mimic with a synthetic, biomimetic lung surfactant formulation is natural lung surfactant's ability to rapidly adsorb to the alveolar air-liquid interface, immediately forming a surface-active layer. A modified PBS run in static mode with a bubble radius of approximately 0.40 mm at 37° C. was used to characterize the adsorption kinetics of the surfactant formulations. FIG. 4 shows the adsorption profile for natural lung surfactant (Natural LS) as well as a synthetic phospholipid mixture alone and with the addition of 1.6 mol % SP-C protein and SP-C analogues. Natural lung surfactant displays very rapid adsorption kinetics, reaching a static surface tension <25 mN/m in less than one minute and a final equilibrium surface tension of ˜23 mN/m. In contrast, the lipid formulation without any protein or peptoid mimimetics displays very poor adsorption characteristics. The lipid formulation fails to reach a static adsorption surface tension lower than ˜53 mN/m even after 20 minutes. The addition of natural SP-C to lipid formulation greatly accelerates the surfactant adsorption to the air-liquid interface, enabling the surfactant film to reach a surface tension of <30 mN/m in less than one minute and a final static surface tension of ˜26 mN/m. The addition of the peptoid analogues to the synthetic lipid formulation similarly improves the adsorption kinetics. All of the peptoid-containing formulations have similar adsorption profiles as the natural LS except for the Mimic C-containing formulation. While the Mimic C formulation only reaches a final surface tension of ˜27 mN/m, this is comparable to the natural SP-C formulation.

Based upon these results, the kinetic adsorption behavior of the lipid formulation is significantly enhanced with the addition of the Nsdp-containing peptoid SP-C mimics. The synthetic formulations not only show comparable adsorption to a natural SP-C-containing surfactant, but also to natural lung surfactant, which contains both SP-B and SP-C proteins. It is somewhat surprising that the Mimic CLeu3 formulation had such a rapid adsorption profile as previous studies with a Nssb-based mimic, containing solely α-chiral, aliphatic residues in the helical region, did not display favorable adsorption. It is believed that the greater bulk and hydrophobicity of the Nsdp residues results in greater structural rigidity and a better ability to perturb and fuse the dispersed surfactant structures to the air-liquid interface. The specific covalent structure of the Nsdp side chain is also likely to be interacting in a specific manner with the lipid acyl chains, as the aromatic-based mimic, Mimic C, was unable to produce as rapid surfactant adsorption despite its likely rigid helical structure.

Dynamic Compression-Expansion Cycling.

To investigate the effectiveness of the peptoid-based SP-C mimic formulations to reduce and control surface tension as a function of surface area, PBS experiments were performed in a dynamic mode at an oscillation frequency of 20 cycles/min and ˜50% reduction in surface area. Features that are important in the compression-expansion loop for biomimetic surfactant film behavior include: (1) the ability to reach a very low surface tension with a small percentage of compression; (2) an ability to respread rapidly, and (3) an ability to control surface tension as a function of bubble surface area with the lowest minimum surface tension possible and a relatively low maximum surface tension. FIG. 3 displays the surface tension as a function of bubble surface area for natural lung surfactant as well as the lipid mixture with and without SP-C (7.3A) and with the peptoid SP-C mimics (7.3B).

Natural lung surfactant is extraordinarily surface-active during the dynamic compression-expansion cycles. The maximum surface tension observed for the natural material is ˜33 mN/m and the minimum surface tension reached upon compression is <1 mN/m. These values are consistent with other studies of natural lung surfactant extracts. The lipid mixture, when used alone; however, exhibits a high maximum surface tension of ˜60 mN/m and a high minimum surface tension of ˜17 mN/m. The lipid formulation is also highly compressible, requiring a significant amount of compression to reach the minimum surface tension. With these properties, the lipid formulation would be a poor SRT. The addition of SP-C to the lipid formulation dramatically improves the surface activity of the film. The SP-C containing film exhibits a maximum surface tension of ˜39 mN/m and a minimum surface tension <1 mN/m during dynamic PBS compression-expansion cycling. The compressibility of the film is also greatly reduced, requiring much less compression to reach a low minimum surface tension.

The addition of the aromatic-based peptoid mimic, Mimic C, to the lipid film also improves the compression-expansion behavior; however, not nearly to the extent of the natural protein (FIG. 5B). The Mimic C film reduces the maximum surface tension of the lipid formulation to a modest 53 mN/m and the minimum surface tension to <1 mN/m. The addition of Mimic CLeu2, containing two aliphatic faces in the helical region, to the lipid formulation results in a dramatic increase in surface activity. The Mimic CLeu2 formulation has a compression-expansion PBS loop that is very similar to the SP-C surfactant with a maximum surface tension of ˜39 mN/m and a minimum surface tension <1 mN/m. Mimic CLeu3 with an entirely aliphatic helical region results in a PBS loop that is further improved. The Mimic CLeu3 formulation exhibits a maximum surface tension of only ˜35 mN/m and a minimum surface tension <1 mN/m. This exceeds the performance of the SP-C-containing surfactant and approaches that of natural lung surfactant. This is extraordinarily surprising as previous studies with peptoid mimics composed of only aliphatic, Nssb residues in the helical region had inferior PBS cycling loops, which required a significant amount of compression to reach a minimum surface tension and the minimum surface tension was occasionally elevated. Also surprising is that Mimic CLeu3 does this in the absence of an N-terminal alkylation motif. Addition of the alkyl chains to the N-terminal region of Mimic CLeu3 further improves the surface activity (FIG. 5B). The alkylated Mimic di-pCLeu3 formulation displays extraordinary surface activity during dynamic cycling with an extremely low maximum surface tension of ˜33 mN/m and a minimum surface tension of <1 mN/m. These values surpass the porcine SP-C formulation and are comparable to the natural lung surfactant formulation, representing the best performing synthetic surfactant formulation studied to date. Therefore, Mimic di-pCLeu3 has great potential for use in a biomimetic SRT for the treatment of nRDS given its relatively facile synthesis and purification as well as its stable secondary structure and extraordinary in vitro surface activity.

Fluorescently Labeled SP-C Mimics in Lipid Bilayers.

To investigate the effect of the peptoid SP-C analogues on the lipid lateral organization and disposition in surfactant-like membranes, GUVs containing a fluorescently labeled SP-C peptide and labeled SP-C peptoid mimics were imaged by confocal fluorescence microscopy. The use of confocal fluorescence microscopy to visualize GUVs is an experimental technique that has recently been used to gain greater insight into the lateral organization and separation of lipid membranes. Surfactant is a unique biomaterial, in that, the coexistence of the segregated lipid-protein domain structures provides a structural basis for its unique surface activity. These small vesicles, ˜20-35 μM diameter, allow the direct visualization of the lipid domain structures during experimentation. FIG. 6 shows the fluorescent images of the lipids alone (panels A and E), with 10 wt % di-pSP-C (panels B and F), with 10 wt % Mimic CLeu3 (panels C and G), and with 10 wt % Mimic di-pCLeu3 (panels D and H). Natural SP-C was not used in this experiment due to the extreme difficulty associated with maintaining its proper secondary structure during the labeling procedure.

The lipid formulation was doped with a lipid probe, DiI_{C18}, which preferentially segregates to the more fluid phase rather than the lipid ordered, gel phase. In the absence of any added peptide or peptoid species, coexistence of fluid and gel phases were observed at room temperature (FIG. 6, panels A and E). The lipid formulation produced stable vesicles with large globular domains of segregated gel-like region (dark regions) that are devoid of the lipid probe (red) and are similar in morphology to lipid monolayer and bilayer films of this formulation at this temperature and estimated surface pressure. Adding the labeled di-pSP-C peptide (yellow) to the lipid film causes a condensation of the solid domains, forming larger, flower-like domain structures in addition to condensed domains that are similar to those with lipid formulation (FIG. 6, panels B and F). The labeled di-pSP-C peptide also preferentially segregates to the less ordered phase as seen by the orange color of the more fluid regions. This is consistent with other studies of SP-C in lipid bilayers where there is a hydrophobic mismatch between the length of the SP-C helix and the DPPC bilayer in the gel phase, causing phase segregation. The di-SP-C peptide also tends to self associated in these regions as evident by the regions of brighter intensity. This is also consistent with previous studies of SP-C at this temperature, where SP-C segregates and self associates at lower temperatures due to the hydrophobic mismatch.

The presence of the peptoid-based SP-C mimics in the lipid bilayers results in morphologies that are have some similarities and differences in comparison to the GUVs containing the SP-C peptide (FIG. 6). Both Mimic CLeu3 and Mimic di-pCLeu3 are closely associated with the lipid bilayer as no fluorescence was observed in the interior of the GUVs. The mimics both preferentially associate with the less ordered lipid phase of the GUVs. Mimic CLeu3 is most similar to the di-pSP-C peptide, in that, this species also segregates and self associates in the fluid regions (FIG. 6, panels C and G). The peptoid helix is estimated to be slightly shorter than an SP-C helix; therefore, it is likely that a greater mismatch between the peptoid helix and the lipid acyl exists. Mimic CLeu3 also appears to have a fluidizing influence on the domain structures as the gel phase domains have coalesced, forming slightly larger domain structures. The addition of Mimic di-pCLeu3 results in a more uniform, fluid phase and does not appear to be strongly self associating (FIG. 6, panels D and H). It is possible that the alkyl chains are assisting in Mimic di-pCLeu3's association with the lipid acyl region, decreasing the tendency for self association and causing the mimic to be more evenly dispersed. This greater dispersion likely contributes to the greater membrane fluidization observed with these results. From these results, it is shown that the morphology induced by this mimic is very similar to GUVs containing both SP-B and SP-C peptide analogues.

To demonstrate the use of such compounds and related compositions, a study was conducted examine the ability of peptoid based SP-B and/or SP-C compositions (termed pB and pC, respectively, for purpose of the study) to mitigate deleterious physiological and biochemical responses associated with ALI. Compositions pB and pC were designed with representative peptoid compounds that mimic the overall hydrophobicity, amphipathicity, and helical structures of SP-B and SP-C, respectively. Compounds selected were N-terminally C₁₋₈ alkylated to mimic the palmitoyl moieties of natural SP-C, a feature known to improve in vitro surface activity. (See, FIG. 7 for an SP-B peptoid and a structurally-related SP-C peptoid.) The hypothesis was that such peptoid enhanced surfactant compositions could demonstrate in vivo efficacy in an animal model of ALI that matches or exceeds that of an animal derived surfactant.

While the extensive alveolar network and capillary vasculature of the pulmonary parenchymal tissue are critical to achieving efficient gas exchange, these delicate structures are highly susceptible to systemic pathogens and environmental toxins. A broad spectrum of direct pulmonary insults and indirect systemic maladies results in lung surfactant deficiency and dysfunction, which leads to ALI. There is currently no cure for ALI, and while exogenous surfactant treatment as part of a multimodal therapy has been shown to mitigate symptoms of the disease for a subset of patients, outcomes of clinical trials have been varied. However, the present invention provides a novel technology platform with characteristics amenable to the treatment of ALI. This inaugural study was designed to investigate the in vivo efficacy of peptoid based surfactants. It demonstrates, using the lung lavage model of ALI—generally-accepted by those skilled in that art as a model for treatment of human subjects, that lung surfactant replacements, in accordance with this invention, can improve physiological and biochemical outcomes to an equivalent or greater extent than treatment with animal derived surfactant. As discussed, below, peptoid enhanced surfactant preparations demonstrated statistically significant, immediate (within 10 minutes of treatment) and/or sustained (10 minutes-2 hours) improvements in PaO₂/FIO₂, shunt fraction, a-A gradient, and PIP. This is an encouraging result for biomimetic surfactants as it marks the first reporting of peptoid enhanced surfactant compositions demonstrating in vivo efficacy.

With reference to examples 8-17, this study evaluated the in vivo efficacy of peptoid based SP-B and SP-C mimics formulated separately and in combination. Tanaka lipids was selected as the lipid carrier for these representative synthetic formulations based on its similarity to the lipid/fatty acid component of natural surfactant and superior in vitro surface activity compared to other lipid formulations. Though the peptoid enhanced surfactants utilized contain a higher amount of protein mimic (˜10 wt %) relative to the quantities of SP-B and SP-C found in extracted surfactant (˜0.5 3 wt % each), this is reasonable because peptoids pB and pC (20 and 22 residues, respectively) represent only a portion of the natural proteins' structures (79 and 34 residues for SP B and SP C, respectively).

The study utilized a lung lavage model of lung surfactant deficiency in adult rats, a model that is well characterized, accepted in the art and has previously been shown to respond to animal derived surfactant preparations. The average heart rate and blood pressure of the various treatment groups showed no notable difference among any of the groups (FIG. 9). The average post lavage decrease in the PaO₂ and increase in PIP showed that pulmonary gas exchange and lung compliance were significantly and uniformly damaged, a condition associated with ALI. Accordingly, as discussed more fully below, this animal model of surfactant deficiency captures most aspects of the pathophysiology associated with ALI (i.e. surfactant alterations) and was deemed well suited for direct comparison of surfactant preparations.

The five physiological responses measured in this study consistently showed that the negative control treatment group (Tanaka lipids) resulted in the least improvement in pulmonary function. FIGS. 8 and 10 show that the Tanaka lipids alone formulation neither achieved the same initial degree of recovery, nor effectively maintained activity throughout the observation period. The notable and consistent improvement in physiological response to peptoid enhanced Tanaka lipid formulations compared to the lipid carrier alone provide evidence for the bioactivity of peptoids to affect improved outcomes using a lung lavage model of ALI.

Whereas all peptoid containing surfactant compositions improved physiological lung function, there were differences in the responses to the individual preparations. The pC composition demonstrated a more significant initial improvement in physiological responses and exhibited sustained benefit throughout the recovery period, compared to the other preparations. The pB composition treatment group, however, consistently had a less significant impact on measured outcomes, and on average appeared only marginally better than Tanaka lipids.

A second observation regarding the responses of the individual preparations lies in comparing the performance of compositions pB and pC formulated separately to that of the pB/pC combination composition. By all physiological measures, the pC treatment group achieved a more favorable outcome than did the pB treatment group. Interestingly, however, the pB/pC group achieved the best sustained response in PaO₂/FIO₂, shunt fraction, and A-a gradient. Without limitation to any theory or mode of operation, variability in the dynamic in vivo environment and lipid composition can influence the extent to which proteins and protein mimics interact. Because the synergistic interaction of protein mimics is dependent on both their chemical structures and the conditions in vivo, it is difficult to generalize observations relevant to a particular system. In this study, however, co dosing pB and pC in the pB/pC composition enabled a better sustained response in some physiological outcomes over the two hour recovery period.

The way in which exogenously administered surfactant is metabolized is another factor that can influence its efficacy. Surfactant delivered to the airspace can subsequently be taken up by alveolar type II cells for recycling or by alveolar macrophages for degradation. In addition, within the airspace, exogenous surfactant can be converted from the active large aggregates to inactive small aggregates. These processes would all impact the efficacy of the exogenous material, and the surfactant pool characterization at the end of the ventilation period provided some insight into these effects. The data showed that the Tanaka lipids treatment group had a larger total surfactant pool than any other group and was statistically different from that of the pC treatment group (FIG. 11A). This difference could be due to disparate surfactant uptake rates for the various surfactant preparations. Because the Tanaka lipid formulation contains no proteins or protein mimics, it is possible that it may not be as readily taken up by type II cells. The rate of conversion from large to small aggregates within the surfactant pool has also been shown, in the literature, to increase under conditions pervasive in an injured lung: 1) increased protease activity, 2) altered surfactant composition, and 3) dynamic changes in surface area due to mechanical ventilation. Injured lungs, therefore, often exhibit an increased amount of total surfactant and a concomitant increase in the less surface active Small Aggregates. FIG. 11B shows that indeed the small aggregate component of the BAL from the Tanaka lipid treatment group was statistically greater than that of any other group. Without limitation, the increase in total surfactant of this group appears to be due to primarily an increase in the less active Small Aggregates.

The results of this in vivo study demonstrate that peptoid enhanced lung surfactant replacements exhibit promising bioactivity and can improve physiological and biochemical outcomes using the lung lavage model of ALI. Such results are significant because biomimetic exogenous lung surfactants afford several advantages over animal derived surfactant replacements. As mentioned above, the high cost of natural surfactant coupled with the large quantities required to treat adults for ALI can make treatment prohibitively expensive. Moreover, the use of a biomimetic surfactant avoids the risk of immune response that is inherent with animal derived products. Biomimetic surfactants of this invention also offer the possibility of a “designer” treatment customized to mitigate specific types of surfactant dysfunction or deactivation induced by the myriad of clinical maladies that result in ALI. Accordingly, additives can be included in a synthetic formulation, not only to improve surface activity, but also to prevent surfactant inhibition, regulate surfactant homeostasis, control inflammatory response and treat bacterial and viral infections (e.g., antibiotic and/or antiviral agents). Finally, peptoids designed as biomimetics specifically exhibit secondary structure that makes them less prone to aggregation, which can result in enhanced shelf life and facilitates synthesis and purification.

Such results, for the first time, demonstrate lung surfactant compositions of this invention, utilizing SP-B and SP-C biomimetic compounds of the sort described herein, can improve physiological and biochemical outcomes to an extent equivalent to or better than animal derived surfactant. While all peptoid enhanced compositions evaluated tended to improve outcomes compared to treatment with the lipid carrier alone, a pC composition exhibited the best and most sustained in vivo response.

### EXAMPLES OF THE INVENTION

The following non-limiting examples and data illustrate various aspects and features relating to the biomimetic lung surfactant compounds, compositions and/or methods of the present invention, including the preparation of various biomimetic SP-C compounds as are available through the synthetic methodologies described herein. In comparison with the prior art, the present compounds, compositions and/or methods provide results and data which are surprising, unexpected and contrary thereto. While the utility of this invention is illustrated through the use of several such compounds and N-substituted glycine residues incorporated therein, it will be understood by those skilled in the art that comparable results are obtainable with various other residue combinations, corresponding lung surfactant compounds and related compositions, as are commensurate with the scope of this invention.

Peptoid synthesis reagents were purchased from Applied Biosystems (Foster City, Calif.) and Sigma-Aldrich (Milwaukee, Wis.). Fmoc-protected amino acids, resins, and di-tert-butyl dicarbonate were purchased from NovaBiochem (San Diego, Calif.). The primary amines and palmitic acid (PA) were purchased from Sigma-Aldrich in the highest purity available. All organic solvents used for sample synthesis, purification, and preparation were HPLC-grade or better and were purchased from Fisher Scientific (Pittsburgh, Pa.). The synthetic phospholipids DPPC and palmitoyloleoyl phosphatidylglycerol (POPG) were purchased from Avanti Polar Lipids (Alabaster, Ala.) and were used as received. Alexa Fluor® 488 carboxylic acid, succinimidyl ester was purchased from Invitrogen (Carlsbad, Calif.). The native SP-C used in these studies was a kind gift from Dr. Perez-Gil and Dr. Ines Plasencia and was extracted from porcine lung surfactant utilizing the methodology of Perez-Gil et al. (Perezgil, J., A. Cruz, and C. Casals, Solubility of Hydrophobic Surfactant Proteins in Organic-Solvent Water Mixtures—Structural Studies on Sp-B and Sp-C in Aqueous-Organic Solvents and Lipids. Biochimica Et Biophysica Acta, 1993. 1168(3): p. 261-270.)

### Example 1

The peptoid-based SP-C mimics shown in FIG. 1 were synthesized on an automated 433A ABI Peptide Synthesizer (Foster City, Calif.) on solid support (Rink amide resin), following a two-step submonomer method as described by Zuckermann et al. (See, Zuckermann, R. N., J. M. Kerr, S. B. H. Kent, and W. H. Moos, Efficient Method for the Preparation of Peptoids Oligo (N-Substituted Glycines) by Submonomer Solid-Phase Synthesis. Journal of the American Chemical Society, 1992. 114(26): p. 10646-10647; and U.S. Pat. No. 6,887,845 each of which is incorporated herein in its entirety.)

Briefly, synthesis was carried out on 0.25 mmol Rink amide resin (NovaBiochem, San Diego, Calif.). After the removal of the first Fmoc protecting group from the resin with 20% piperidine in N,N-dimethylformamide (DMF) and rinsing of the resin with DMF, the monomer addition cycle was performed by first acetylating the resin with the addition of 1.2 M bromoacetic acid in DMF, followed by N,N-diisopropyl carbodiimide (DIC). The acetylation step was carried out for 45 minutes and then the resin was washed with DMF. The resin-bound halogen was then displaced by 1.0 M primary amine submonomer in N-methylpyrrolidinone (NMP), which was added to the resin and allowed to react for 90 minutes. (The N-substituent of a particular primary amine corresponds to the N-substituent of a glycine residue within a resulting peptoid sequence. Accordingly, as would be understood in the art, N-substituent identity is limited only by the synthetic or commercial availability of a corresponding primary amine and use thereof in peptoid preparation.) The two-step cycle was repeated until the desired length and sequence of the peptoid was obtained, except for the addition of the lysine-like submonomer (N_{Lys}), the alkyl submonomers (e.g., N_{oct}), and the proline residue. The displacement step for the Boc-protected N_{Lys}, submonomer and the N_{oct} submonomers was extended to 120 minutes while for the addition of the proline residue, a PyBrop activating system was employed. Additionally, due to poor solubility in NMP, the N_{oct} submonomer was dissolved at 0.8 M in dichloromethane:methanol (1:1). After the proline addition, the Fmoc group was removed with piperidine as before and the peptoid cycle was continued.

Peptoid oligomers were cleaved from the resin and deprotected with 90% TFA along with necessary scavengers for 5 minutes. The crude products were then purified by preparative HPLC on a Waters system with a Vydac C4 column and a linear gradient of 50-90% solvent B in solvent A over 70 minutes (solvent A=0.1% TFA in water and solvent B=0.1% TFA in isopropanol). The final purity of the peptoids was confirmed by reversed-phase HPLC to be >97%. Electrospray mass spectrometry was used to confirm correct molar masses of the peptoids.

### Example 2

Native lung surfactant was obtained from freshly slaughtered ovine lungs (Chiappetti Lamb and Veal, Chicago, Ill.) following procedures previously reported. (Notter, R. H., J. N. Finkelstein, and R. D. Taubold, Comparative Adsorption of Natural Lung Surfactant, Extracted Phospholipids, and Artificial Phospholipid Mixtures to the Air-Water-Interface. Chemistry and Physics of Lipids, 1983. 33(1): p. 67-80.) (Whitsett, J. A., B. L. Ohning, G. Ross, J. Meuth, T. Weaver, B. A. Holm, D. L. Shapiro, and R. H. Notter, Hydrophobic Surfactant-Associated Protein in Whole Lung Surfactant and Its Importance for Biophysical Activity in Lung Surfactant Extracts Used for Replacement Therapy. Pediatric Research, 1986. 20(5): p. 460-467.) (Ingenito, E. P., L. Mark, J. Morris, F. F. Espinosa, R. D. Kamm, and M. Johnson, Biophysical characterization and modeling of lung surfactant components. Journal of Applied Physiology, 1999. 86(5): p. 1702-1714.) Briefly, excised lungs with trachea and bronchi intact were lavaged with ˜2 liters of cold (4° C.) 0.15 M NaCl under 25 cm H₂O of pressure within 1 hour of tissue procurement. After instillation of the saline solution, the filled lungs were elevated and the return collected by passive drainage. Collected lavage material showing any signs of contamination with blood was discarded. Blood-free lavage fluid was then pooled and centrifuged at 150×g at 4° C. for 10 minutes to remove any cellular debris. The supernatant containing the surfactant material was retained and pooled again. The crude surfactant was then harvested by medium-speed centrifugation at 20,000×g at 4° C. for 45 minutes. The pelleted surfactant was resuspended in 0.15 M NaCl and dispersed by injection through a syringe fitted with a 22-gauge needle. The resuspended material was then layered over 0.8 M sucrose in 0.15 M NaCl and centrifuged at 30,000×g at 4° C. for 45 minutes using a swinging bucket rotor. Pellicles at the interface were aspirated, pooled, and resuspended in 0.15 M NaCl as before. The collected material was then ultracentrifuged at 60,000×g at 4° C. for 30 minutes to wash and concentrate the whole surfactant. The supernatant from this step was discarded and the pelleted material was resuspended in a small amount of 0.15 M NaCl.

The surfactant lipids and the hydrophobic proteins were then extracted from the isolated whole surfactant by the method of Bligh and Dyer. (Bligh, E. G. and W. J. Dyer, A Rapid Method of Total Lipid Extraction and Purificatiion. Canadian Journal of Biochemistry and Physiology, 1959. 37(8): p. 911-917.) The extracted surfactant dissolved in the organic solvent was dried under a stream of nitrogen and then placed under high vacuum overnight. The fully dried material was then reconstituted by adding a small amount of aqueous buffer (0.15 M NaCl, 5 mM CaCl₂, and 10 mM HEPES at pH 6.90) and aspirated through a 22-gauge needle. The lipid concentration of the purified surfactant was then quantified using the method of Stewart while the protein content was determined using a BCA protein assay kit (Thermo Fisher Scientific, Rockford, Ill.).

### Example 3

A di-alkylated peptide mimic of SP-C (di-pSP-C) was used for the giant unilamellar vesicle (GUV) studies as the labeling of native SP-C is quite problematic due to the unstable secondary structure. di-pSP-C was synthesized on a 0.25 mmol scale on an Applied Biosystems 433A automated peptide synthesizer, using standard Fmoc chemistry, and a prederivatized low-loading, Wang-Leu resin except for the acetylation and deprotection steps in which dimethyl sulfoxide was used as the solvent during acetylation and a 4% (v/v) 1,8-diazabicyclo-[5.4.0]undec-7-ene:piperidine (1:1) in DMF solution was used for deprotection. Cleavage and deprotection of the peptide-resin was carried out by mixing with aqueous 90% TFA and necessary scavengers for 2 hours. The reaction mixture was then immediately filtered, diluted with isopropanol and water, frozen, and lyophilized to yield the crude peptide. The crude material was then dissolved with hexafluoroisopropanol and isopropanol.

The crude SP-C peptide was purified by preparative HPLC on a Waters system with a Vydac C4 column and a linear gradient of 60-100% solvent B in solvent A over 60 minutes (solvent A=0.1% TFA in water and solvent B=0.1% TFA in isopropanol). The final purity of the protein was confirmed by reversed-phase HPLC to be >97%. Electrospray mass spectrometry was used to confirm correct molar mass.

### Example 4

Fluorescently labeled di-pSP-C and SP-C peptoid mimics were prepared by labeling the peptide in organic solvent as described previously in the literature. (Plasencia, I., A. Cruz, J. L. Lopez-Lacomba, C. Casals, and J. Perez-Gil, Selective labeling of pulmonary surfactant protein SP-C in organic solution. Analytical Biochemistry, 2001. 296(1): p. 49-56.) Briefly, ˜2 mg each of the purified peptide and peptoids were dissolved in a small amount of methanol, ˜3 mL. The apparent pH of the peptide and peptoid solutions was adjusted to ˜7.0 by adding an appropriate volume of 50 mM triethylamine in methanol. The pH adjustment is necessary so as to preferentially deprotonate the N-terminal amine group, allowing for more specific labeling. The peptide and peptoid solutions were then separately incubated in total darkness at 4° C. with constant stirring in the presence of Alexa Fluor® 488 carboxylic acid, succinimidyl ester. The reaction was stopped after 12 hours by the addition of 2 M HCl until the pH decreased below 3. The unreacted probe was then immediately separated from the labeled compounds by reverse-phase HPLC on a Jupiter C18 column (Phenomenex, Torrance, Calif.) using a gradient of 30-90% solvent B in solvent A over 40 minutes (solvent A=0.1% TFA in water and solvent B=0.1% TFA in isopropanol). The isolated labeled compounds were collected in tared vials and lyophilized to remove excess solvent.

### Example 5

CD measurements were performed on a Jasco model 715 spectropolarimeter (Easton, Md.). Stock solutions for dilution to appropriate concentrations for CD were made immediately before analysis in tared vials by precise weighing of added solvent and at least 2 mg of lyophilized peptoid powder, to produce a sample of accurately known concentration. The peptoid samples initially in methanol were diluted with additional methanol to a CD sample concentration of ˜60 μM. CD spectra were acquired in a quartz cylindrical cell (Hellma model 121-QS, Forest Hills, N.Y.) with a path length of 0.02 cm, employing a scan rate of 100 nm/min between 185-280 nm with 0.2 nm data pitch, 1 nm bandwidth, 2 second response, 100 mdeg sensitivity, and 40 successive spectral accumulations. Data are expressed in terms of per-residue molar ellipticity (deg cm²/dmol), as calculated per mole of amide residues and normalized by the molar concentration of the peptoid.

### Example 6

Static and dynamic characterization of surfactant film properties were performed on a modified PBS (General Transco, Largo, Fla.) as described in the literature, in which an imaging system is employed to accurately track bubble size and shape throughout the experiment. (Seurynck, S. L., N. J. Brown, C. W. Wu, K. W. Germino, E. K. Kohlmeir, E. P. Ingenito, M. R. Glucksberg, A. E. Barron, and M. Johnson, Optical monitoring of bubble size and shape in a pulsating bubble surfactometer. Journal of Applied Physiology, 2005. 99(2): p. 624-633.) Briefly, the lipid mixture (DPPC:POPG:PA, 68:22:9 (by weight)) was dissolved in chloroform:methanol (3:1) and spiked with 1.6 mol % SP-C protein or peptoid mimic, analogous to 10 wt % SP-C. (Tanaka, Y., T. Takei, T. Aiba, K. Masuda, A. Kiuchi, and T. Fujiwara, Development of Synthetic Lung Surfactants. Journal of Lipid Research, 1986. 27(5): p. 475-485.) The samples were prepared in Eppendorf tubes, dried under vacuum, and resuspended at room temperature in an aqueous buffer solution (0.15 M NaCl, 5 mM CaCl₂, and 10 mM HEPES at pH 6.90) to a phospholipid concentration of 1.0 mg/mL. Samples were then mixed with a pipette 20 times, sonicated with a probe sonicator for 15 seconds twice, and then mixed again 20 times with a pipette. Samples were then loaded into the sample chamber using a modified leak-free methodology, as described in the literature. The sample chamber was placed in the PBS at a temperature of 37° C. A bubble with a radius of 0.4 mm was then formed and an imaging acquisition system was used to determine the bubble size. Trans-film bubble pressure was recorded as a function of time while holding bubble radius static for 20 minutes during static adsorption experiments. Dynamic measurements of surface tension as a function of bubble surface area were subsequently collected by cycling the bubble radius between approximately 0.4 mm and 0.55 mm at an oscillation frequency of 20 cycles/min for 10 minutes.

### Example 7

GUVs of the synthetic lung surfactant mixtures were prepared as previously described. (de la Serna, J. B., J. Perez-Gil, A. C. Simonsen, and L. A. Bagatolli, Cholesterol rules—Direct observation of the coexistence of two fluid phases in native pulmonary surfactant membranes at physiological temperatures. Journal of Biological Chemistry, 2004. 279(39): p. 40715-40722.) Briefly, 0.5 mol %, of a fluorescently labeled lipid, DiI_{C18} (Sigma Aldrich, Milwaukee, Wis.) along with 10 wt % of the appropriate peptide or peptoid labeled with Alexa Fluor® 488 and dissolved in methanol was added to a 0.5 mg/mL lipid mixture (DPPC:POPG:PA, 68:22:9 (by weight)) dissolved in chloroform:methanol (3:1).

The GUVs were then formed using a electroformation method as described in the literature by Angelova and Dimitrov. (Angelova, M. I. and D. S. Dimitrov, Liposome Electroformation. Faraday Discussions, 1986: p. 303-+.) Approximately 3 μL of the labeled lipid or lipid and peptide/peptoid solution was spread onto the surface of a platinum wire electrode in a sample well of a specially designed Teflon chamber. The chamber was then placed under vacuum in darkness for at least 1.5 hours to evaporate the trace solvent. After evaporation of the solvent, 500 mL of a 200 mOsM sucrose solution was added to each sample well and a low frequency AC field of 10 Hz and 1.5 V amplitude was applied for 1.5 hours using a function generator. A circulating water bath was also used during this process to maintain the temperature of the sample chamber at 60° C., which is sufficiently above the fluid/gel transition temperature. The resulting GUVs were then harvested with a pipette and transferred to a plastic vial.

Just prior to GUV imaging, 50 μL aliquots of the harvested GUV suspension was transferred into a single well of an eight-well cell culture plate (Lab-Tek Brand Products, Naperville, Ill.) containing 200 μL equi-osmolar glucose in each well. Due to the density difference between the sucrose and glucose, the suspended GUVs settled on the bottom of the plate. The GUVs were then imaged at room temperature, 21° C., using an inverted confocal microscope (Zeiss LSM 510 META). The excitation wavelengths were 543 nm for the DiI_{c18} and 488 nm for the Alexa Fluor® 488 labeled compounds.

Previous research with synthetic peptide constructs of SP-C has shown that the overall secondary structure of this region rather than the exact side chain chemistry is more pertinent to its surface activity in phospholipid films. A similar finding with some of the peptoid SP-C analogues was also observed. However, in addition to the rigid secondary structure, the specific side chain chemistry is also important in promoting favorable interactions between the SP-C mimics and the phospholipids. As shown by the present invention, it can be beneficial to include both α-chiral, aromatic and α-chiral, aliphatic residues to preserve, respectively, SP-C rigidity and side chain structure. Such results can be shown, for instance, by utilizing varying amounts of a bulkier, more rigid aliphatic side chain, Nsdp.

Examples 8-17, in particular, can be considered in conjunction with in vivo treatments and related methodologies of this invention.

### Example 8

Peptoid Synthesis and Purification. Peptoids were synthesized using an ABI 433A peptide synthesizer (Foster City, Calif.) on Rink amide resin (NovaBiochem, San Diego, Calif.) as described, above, using peptoid synthesis reagents (Sigma-Aldrich (Milwaukee, Wis.), solvents (Fisher Scientific (Pittsburgh, Pa.), and side chain primary amines benzylamine, octadecylamine, isopropylamine, isobutylamine, S-alpha methylbenzylamine, N-tert-butoxycarbonyl-1,4 diaminobutane, and L-proline. Resin-bound peptoids were cleaved in an trifluoroacetic acid scavenger mixture and purified by standard reversed phase HPLC (RP-HPLC) (C4 or C18 column, linear acetonitrile/water gradient). Analytical RP HPLC purity was >97%; and molar masses were confirmed using electrospray ionization mass spectrometry (ESI/MS).

### Example 9

Preparation of Surfactant Mixtures. Dipalmitoyl phosphatidylcholine (DPPC) and palmitoyoleol phosphatidylglycerol (POPG) (Avanti Polar Lipids, Alabaster, Ala.), palmitic acid (PA) (Sigma Aldrich), and solvents (Fisher Scientific) comprised the Tanaka lipid formulation (22.5 mg Tanaka lipids per animal), which was prepared in a glass vial by combining individual lipid stock solutions to yield a 68:22:9 (w:w:w) mixture of DPPC:POPG:PA in chloroform/methanol (3:1 v:v) solution. Peptoid was added to the lipids from methanol stock solutions at ˜2 mol % peptoid (˜10 wt % relative to total lipid content), and in two peptoid formulations, 1 mol % per peptoid. Surfactant mixtures were dried under nitrogen, lyophilized, and stored at 20° C. Adding sterile saline (25 mg/mL) and resuspending provided a homogenous, flowable lipid peptoid surfactant composition.

### Example 10

Animal Experiments. Procedures were approved by AUS at the University of Western Ontario, London Ontario, Canada, according to the CCAC. Sprague Dawley rats (360-410 g) (Charles River, St. Constant, PQ, Canada) were weighed, anesthetized by intraperitoneal (i.p.) injection (75 mg/kg Ketamine Hydrochloride, 5 mg/kg Xylazine, sterile 0.15 M saline), and given 0.05-0.1 mg/kg of buprenorphine subcutaneously. Right carotid artery access via isolation and cannulation permitted blood gas sampling, measuring vital signs, and instilling fluids (0.15 M saline, 0.5 mL/kg/hour), while similarly obtained jugular venous line access allowed administering of ˜1.0 mg/kg/min Propofol and fluids. Following tracheostomy and endotracheal tube placement, pancuronium bromide (1 mg/kg i.v.) was administered to inhibit spontaneous movement. Ventilator settings were: tidal volume, 8 mL/kg; positive end expiratory pressure (PEEP), 5 cm H₂O; respiratory rate, 55-60 breaths/minute; and FiO₂, 1.0 (volume cycle mechanical rodent ventilator, Harvard Instruments, St. Laurent, PQ, Canada; airway pressure monitor, Caradyne Ltd, Indianapolis, Ind.). Initial inclusion criterion was baseline PaO₂>400 mmHg.

Whole lung lavage was performed. After ventilator removal, 0.15 M NaCl (10 mL, 37° C.) was instilled/withdrawn from the lungs, followed by mechanical ventilation. Lungs were lavaged four times, 5 minutes apart preceding a blood gas measurement. Study inclusion required PaO₂<120 mmHg. Non inclusive animals were re-lavaged until the inclusion criterion was satisfied.

Animals were randomized into five treatment groups: 1) bovine lipid extract surfactant (BLES, BLES Biochemicals, London Ontario, Calif.) (positive control, n=7), 2) surfactant composition pB (n=6), 3) surfactant composition pC (n=7), 4) surfactant composition pB/pC (n=7), 5) Tanaka lipids (negative control, n=5). After ventilator removal, upright animals were instilled with a 50 mg/kg surfactant bolus endotracheally via syringe, and then a 3 mL air bolus that ensured distribution to distal regions. Ventilation and monitoring occurred for 2 hours, with blood gas sampling at semi regular timepoints. Recovery vital signs were monitored for adequate perfusion, as was anesthestic state. Measured physiological responses included PaO₂, blood pH, shunt fraction, A-a gradient, and peak inspiratory pressure (PIP). Post-experiment, animals were euthanized via sodium pentobarbital, exsanguinated, the chest wall opened, lung-lavaged five times, and total lavage volume was recorded.

### Example 11

Surfactant Analysis. After broncheoalveolar lavage fluid (BAL) centrifugation (150 g, 10 minutes), 5 mL of supernatant “Total Surfactant (TS)” was aliquoted for further analysis, and the remainder centrifuged (40,000 g, 15 minutes) to separate supernatant Small Aggregates (SA) from pellet. Resuspended pellets (in 2 mL saline) produced Large Aggregates (LA). Aliquots were extracted (Bligh/Dyer method), phospholipids quantified (Duck Chong phosphorous assay, Duck Chong CG. Rapid sensitive method for determining phospholipid phosphorus involving digestion with magnesium nitrate. Lipids 1979; 14:492 497), and BAL total protein content determined (micro BCA protein assay, Pierce Biotechnology, Rockford, Ill.).

### Example 12

Statistical Analysis. Presented data are means±SEM and were analyzed via one way ANOVA using the Tukey-Kramer method (p<0.05).

### Example 13

Physiological Responses. In general, the surgical procedure was well tolerated by the animals. Three animals died during the procedure, and 13 animals did not meet the study inclusion criteria. For the 32 animals included in the study, the average baseline blood oxygen level (PaO₂), normalized to the fraction of inspired oxygen (FIO₂) (1.0 throughout all experiments), of 435.7±4.9 mm Hg was reduced to 88.3±2.5 mm Hg post lavage, reflective of surfactant deficiency (FIG. 8A). Similarly, the average baseline peak inspiratory pressure (PIP) of 12.0±0.3 cm H₂O was increased to 20.8±0.4 cm H₂O post-lavage. (The average blood pressure and heart rate for each treatment group over the time course of the experiments are shown in FIGS. 10A-B.)

### Example 14

The average PaO₂/FIO₂ as a function of time is shown for each treatment group in FIG. 8A. The immediate response to surfactant treatment is reflected in changes from the pre-treatment (Pre Rx) condition to the 10 minute data time point. The ability of surfactant treatments to sustain a response was gleaned by comparing the 10 minute time point data to conditions at the end of the two hour observation period. Animals treated with the positive control BLES (p<0.001), pC (p<0.0005), and pB/pC (p<0.007) showed a statistically significant, immediate improvement in oxygenation upon treatment. The immediate improvements demonstrated by the negative control Tanaka lipids group (p<0.23) and pB group (p<0.07) were not significant. Treatment with BLES (p<0.16), pC (p<0.15), and pB/pC (p<0.11) also demonstrated better sustained oxygenation throughout the two hour observation period compared with the pB (p<0.47) and Tanaka lipid (p<0.60) treatment groups. Animals in the pC treatment group were correlated with the highest arterial blood oxygenation levels throughout the study.

### Example 15

The blood pH as a function of time is shown for each treatment group in FIG. 8B. Comparing baseline to pre treatment conditions, pulmonary lavage caused a significant (p<0.005) and uniform lowering of the blood pH in all treatment groups. On average, the highest blood pH outcome was achieved by the BLES treatment group, which was found to be statistically different (p<0.05) from the Tanaka lipids treatment group at t>45 minutes. Among peptoid enhanced surfactants, treatment with pC exhibited the most complete return to baseline conditions; the blood pH of this treatment group was statistically different (p<0.05) from the Tanaka lipids treatment group at 75 and 105 minutes.

### Example 16

FIG. 10 displays three additional indicators of pulmonary function, including shunt fraction, A-a gradient, and PIP. Ten minutes after treatment, the shunt fraction decreased significantly for the BLES (p<0.0005), pC (0.001), and pB/pC (p<0.01). The further decrease in shunt fraction observed from the 10 minute time point until the end of the two hour observation period was statistically significant for the pC (p<0.05) and pB/pC (p<0.05) treatment groups. As shown in FIG. 10A, the pC treatment group was shown to be statistically different (p<0.05) from the pB and Tanaka lipids treatment groups at selected timepoints. The A-a gradient data shown in FIG. 10B exhibits a statistically significant, immediate response for all treatment groups (p<0.05) except Tanaka lipids (p<0.20). The pC treatment group resulted in the most significant immediate decrease (p<0.0003), but the pB/pC treatment group resulted in the best sustained response from the 10 minute time point throughout the observation period (p<0.1). The PIP data shown in FIG. 10A demonstrated statistically significant immediate improvement at 10 minutes for BLES (p<0.05) and pC (p<0.01) (pB and pB/pC exhibited p<0.1). The Tanaka lipid treatment group immediate improvement was not significant (p<0.2). No treatment groups demonstrated sustained improvement of PIP throughout the 2 hour observation period.

### Example 17

Surfactant Pool Evaluation. Since the efficacy of surfactant may be influenced by its metabolism within the airspace, phospholipid pools and total protein content in the broncheoalveolar lavage (BAL) fluid from each animal were evaluated at the end of the ventilation period. The average amount of total surfactant (TS), large aggregates (LA), and small aggregates (SA) obtained from the BAL of each treatment group is shown in FIG. 11A. While there was no statistically significant difference between the large aggregate contents of the treatment groups, the amount of small aggregates was higher in the Tanaka lipid treatment group than in any other (p<0.05). The average amount of total surfactant was highest for the Tanaka lipids treatment group, and statistically higher (p<0.05) than the pC treatment group. The average total protein content of the BAL for each treatment group is shown in FIG. 11B. The data show that there was no statistically significant difference in the total protein content among the various treatment groups.

As discussed above, CD was used to characterize the peptoid analogues' secondary structures in solution. All the peptoid analogues were found to replicate SP-C's helical secondary structure in solution. The spectral intensities of the Nsdp-containing analogues were greater than those observed for previous α-chiral, aliphatic containing mimics, indicating increased helical propensity. The structures were also stable over time, which is a major advantage over the metastable natural SP-C protein. When combined with a biomimetic phospholipid formulation, the SP-C peptoid analogues significantly improved both the static and dynamic surfactant properties on a PBS instrument and were very similar to native, porcine SP-C. The peptoid mimic containing solely α-chiral, aliphatic residues in the helical region were observed to exhibit the greatest in vitro surface activity. The bulkier Nsdp side chain is believed to result in a peptoid SP-C helix that is both structurally rigid and biomimetic. Adding two amide-linked alkyl chains, which mimic the palmitoyl chains of SP-C, further improved the PBS surface activity to comparable levels as natural lung surfactant containing both SP-B and SP-C proteins. The lateral organization of lipid bilayers and the disposition of the peptoid analogues in these films were also investigated by confocal fluorescence microscopy of GUVs. In the GUVs, it is found that the non-alkylated, aliphatic mimic has a similar influence on the lipid domain structures as a peptide SP-C mimic and that the non-alkylated peptoid mimic is also prone to self association in the more fluid and uniform regions. However, alkylating the mimic, results in a more fluid morphology and a lesser propensity to self associate.

Taken together, these studies show that both the specific side chain chemistry and the helical rigidity can be used to affect biophysical activity of peptoid-based SP-C mimics. The increased aliphatic content likely provides favorable interactions with the lipid acyl chains and better mimics the native protein. In fact, while the in vitro surface activity of an alkylated peptoid mimic approaches that of natural lung surfactant, the aforementioned pC composition showed, from several perspectives, in vivo activity equivalent to or better than animal-derived surfactant. These studies are very promising for the development of a fully functional, biomimetic surfactant therapy for the treatment of respiratory-related disorders. Specifically, a biomimetic lung surfactant formulation including the aforementioned peptoid-based mimics of SP-B could be used for treatment of or as a supplement in treatment of IRDS, ARDS, meconium aspiration syndrome, pneumonia, sepsis, lung injury, bronchopulmonary dysplasia, asthma, cystic fibrosis, idiopathic interstitial pneumonias, tuberculosis, and other bacterial and/or viral infections of the lung.

While the principles of this invention have been described in connection with specific embodiments, it should be understood clearly that these descriptions are added only by way of example and are not intended to limit, in any way, the scope of this invention. For instance, any one or more of the peptoid compounds of this invention can be prepared, as described herein, to provide alternate hydrophobic components incorporating other residues, side-chain moieties and/or residue sequences for helical conformation. Likewise, one or more alternate amino acid or N-substituted glycine residues can be introduced to the N-terminal component. Other structural and/or functional variations of the present peptoid compounds will be understood by those skilled in the art and made aware of this invention.

Likewise, any one or more of the peptoid compounds of this invention can be incorporated into a lung surfactant composition, such compositions as can optionally comprise one or more synthetic or naturally derived surfactants proteins, lipids and/or fatty acids. While several such compositions are formulated as described herein, it will be understood by those skilled in the art that such formulations and effective dosages or concentrations are limited only by sufficient administration and corresponding treatment of a pulmonary disorder. An effective dosage will be understood by those skilled in the art and can be determined in accordance with the guidelines/parameters and indications demonstrated herein. Generally, administration can be tracheally, local or as otherwise designed to target an alveolar network and/or a corresponding air/liquid interface. Accordingly, compositions of this invention can be formulated as part of a solution, an emulsion, a suspension, a bolus and the like for delivery and/or administration by deposition, injection, aerosol spray or any other technique known in the art. Other formulations and/or delivery techniques, for the present compositions, will be understood by those skilled in the art made aware of this invention.

We claim:

1. A poly-N-substituted glycine compound of a formula
A-X—Y—Z—B
wherein A is an N-terminus selected from H and 1-2 N-alkyl substituted glycine residues, where each said alkyl substituent is independently selected from about C₄— about C₂₄ linear, branched and cyclic alkyl moieties; X is a component comprising residues selected from 1-about 7 N-substituted glycine residues and proline, and combinations thereof; Y is a component comprising 1-about 6 N_{Lys} residues; Z is a component comprising about 12-about 20 N-substituted glycine residues, said residues comprising residues selected from Nₛₚₑ, N_{sdp} and N_{ssb} residues, and combinations thereof; and B is a C-terminus selected from NH₂ and 1-2 N-substituted glycine residues selected from N_{sdp} and Nₛₚₑ.

2. The compound of claim 1 wherein A is a sequence of two N-alkyl substituted glycine residues.

3. The compound of claim 2 wherein each said alkyl substituent is independently selected from linear C₈-C₂₀ alkyl moieties.

4. The compound of claim 1 where said X component is a sequence of 3-5 residues.

5. The compound of claim 4 wherein said X component comprises at least one Nₚₘ residue and at least one proline residue.

6. The compound of claim 1 wherein said Z component is a sequence of about 10-about 16 residues selected from Nₛₚₑ and N_{sdp} residues.

7. The compound of claim 6 wherein said Z component is a sequence of about 12-about 14 N_{sdp} residues.

8. The compound of claim 1 wherein each said X and Z components independently comprise at least one other residue selected from proline and N-substituted glycine residues, said N-substituents independently selected from α-amino acid side chain moieties and carbon homologs thereof.

9. The compound of claim 8 wherein X and Z independently comprise residues selected from Nₚₘ, N_{ssb}, N_{sdp}, N_{Leu}, N_{Ile}, Nₚₕₑ, N_{Trp}, N_{His} and N_{Tyr}, and combinations thereof.

10. The compound of claim 1 wherein said Y component is a sequence of 2-3 N_{Lys} residues.

11. The compound of claim 1 wherein Y comprises an N_{Arg} residue.

12. The compound of claim 1 wherein said X component is a sequence of 3-5 residues, said Y component is a sequence of 2-3 residues, said Z component is a sequence of 10-14 residues and B is NH₂.

13. The compound of claim 1 in a lung surfactant composition.

14. A poly-N-substituted glycine compound of a formula
A-X—Y—Z—B
wherein A is an N-terminus selected from 1-2 N-alkyl substituted glycine residues, where each said alkyl substituent is independently selected from about C₄— about C₂₄ linear, branched and cyclic alkyl moieties; X is a component comprising residues selected from about 3-about 5 N-substituted glycine residues and proline, and combinations thereof; Y is a component comprising about 2-about 3 N_{Lys} residues; Z is a helical component comprising about 10-about 14 N-substituted glycine residues, said residues comprising residues selected from Nₛₚₑ and N_{sdp} residues, and combinations thereof; and B is a C-terminus selected from NH₂ and 1-2 N-substituted glycine residues selected from N_{sdp} and Nₛₚₑ.

15. The compound of claim 14 wherein A is a sequence of two N-alkyl substituted glycine residues.

16. The compound of claim 15 wherein each said alkyl substituent is independently selected from linear C₈-C₂₀ alkyl moieties.

17. The compound of claim 14 wherein said X component comprises at least one Nₚₘ residue and at least one proline residue.

18. The compound of claim 14 wherein said Z component is a sequence of residues selected from Nₛₚₑ and N_{sdp} residues.

19. The compound of claim 18 wherein at least about ⅔ of said Z component residues are N_{sdp} residues.

20. The compound of claim 14 wherein each said X and Z components independently comprise at least one other residue selected from proline and N-substituted glycine residues, said N-substituents independently selected from α-amino acid side chain moieties and carbon homologs thereof.

21. A lung surfactant composition comprising a biomimetic SP-C compound of claim 1, and a lipid component selected from naturally-derived phospholipids, non-natural phospholipids, commercial surface-active agents and combinations thereof.

22. The composition of claim 21 comprising a compound of claim 14.

23. The composition of claim 21 wherein X and Z independently comprise residues selected from Nₚₘ, N_{ssb}, N_{sdp}, N_{Leu}, N_{Ile}, Nₚₕₑ, N_{Trp}, N_{His} and N_{Tyr}, and combinations thereof.

24. The composition of claim 21 wherein said lipid component comprises DPPC, POPG and PA.

25. The composition of claim 21 comprising a component selected from naturally-derived SP-B components, biomimetic SP-B components and combinations thereof.

26. The composition of claim 22 wherein said biomimetic SP-C compound and a said SP-B component comprise up to about 20 wt. % of said composition.

27. A method of treating a mammalian pulmonary disorder, said method comprising:
providing a mammalian subject exhibiting a physiological condition comprising a lung surfactant deficiency; and
administering a composition of claim 21 to said subject, said composition in a therapeutically effective amount.

28. The method of claim 27 wherein said composition comprises a component selected from naturally-derived SP-B components, biomimetic SP-B components and combinations thereof.

29. The method of claim 28 wherein said biomimetic SP-C compound and a said SP-B component comprise up to about 20 wt. % of said composition.

30. The method of claim 28 wherein said composition comprises a compound of claim 14.

31. The method of claim 27 wherein said composition is formulated as a liquid bolus or an aerosol spray.

32. The method of claim 27 wherein said composition is administered via the trachea of said subject.
